(19) United States
(12) Patent Application Publication
(10) Pub. No.: US 2003/0132399 A1
(43) Pub. Date: Jul. 17, 2003

(54) Method and apparatus for laser processing

(76) Inventors: Stefan Dietrich, Karlsruhe (DE); Juergen Pflaum, Bruchsal (DE)

Correspondence Address: HARNESS, DICKEY & PIERCE, P.L.C., P.O.BOX 8910, RESTON, VA, 20195, US

(21) Appl. No.: 10/345,170

(22) Filed: Jan. 16, 2003

(30) Foreign Application Priority Data
Jan. 16, 2002 (DE) 10201477.9

(51) Int. Cl. A61N 5/00; G21G 5/00

(52) U.S. Cl. 250/492.1

(57) ABSTRACT

A method and an apparatus are for processing objects by using laser beams. In this case, a primary laser beam emitted by a laser is alternately split by a so-called chopper element into at least two component beams and with spatially different beam paths. Each of the two component beams and is respectively guided onto the object to be processed by a deflecting unit and, respectively. The temporal beam splitting of the primary laser beam into the at least two component beams has the advantage that in each case the deflecting unit, respectively, that is not used at a specific instant, since the other laser beam is actually being used to process the object, can be brought into a new predetermined position such that the subsequent laser beam led via this deflecting unit strikes the desired subregion of the object without a time delay.

The present application hereby claims priority under 35 U.S.C. §119 on German patent application number DE 10201477.9 filed Jan. 16, 2002, the entire contents of which are hereby incorporated herein by reference.

### FIELD OF THE INVENTION

The invention generally relates to a method and an apparatus for processing an object by means of laser beams.

### BACKGROUND OF THE INVENTION

Material processing by using laser beams has become increasingly important in recent years owing to the rapid development of laser technology. Particularly in the electronics fabrication sector, the increasing miniaturization of the components has turned laser processing of printed circuit boards and substrates into an indispensable tool, rendering possible the microstructuring of components and/or substrates that is required because of the miniaturization of the components. Thus, for example, it is possible to bore into printed circuit boards and substrates holes which have a diameter that is substantially smaller by comparison with the hole diameters of holes bored using conventional drills. Assuming that the laser power of the laser beam striking a printed circuit board is precisely known, it is possible in addition to through holes also to bore so-called blind holes which are important, in particular, for multilayer printed circuit boards, since various metal layers of the multilayer printed circuit board can be connected to one another in an electrically conducting fashion by a subsequent metallization of a blind hole.

In many applications, objects are processed not on the entire object surface but only in individual subregions. In such cases, the object to be processed can be processed speedily by use of a single laser processing device that has a single laser whose emitted laser beam is split by a conventional beam splitter into two component beams. These two component beams are guided via two individually drivable deflecting units onto the different subregions of the object to be processed. Semi-transmitting mirrors are generally used as beam splitters, as a rule. The power splitting between the two component beams is determined by the reflection and transmission coefficients of the beam splitter used. However, it is not possible for these coefficients to be determined exactly even given high quality beam splitters, and so it is very difficult to produce a beam splitter with a reflection or transmission coefficient of exactly 50%. Consequently, the use of a beam splitter to produce two component beams generally entails nonuniform power splitting and therefore a different quality of the structures processed with different component beams.

### SUMMARY OF THE INVENTION

It is therefore an object of an embodiment of the invention to create a method and an apparatus for processing objects by use of laser beams, two component beams with exactly the same optical power being produced with the use of a single laser for object processing.

This object may be achieved by a method and by an apparatus for processing objects by use of laser beams.

Two or more further subregions with substantially reflecting properties may be used for the purpose of producing two or more reflected component beams. A spatial separation of the reflected component beams presupposes in this case that the further subregions are arranged at an angle to one another in each case. It may be pointed out that it is possible in principle in this way to produce as many reflected component beams as desired.

During the processing of a specific region of the object to be processed, the deflecting units not participating in the object processing may be adjusted in such a way that the subsequently produced component beams required for the further object processing are guided exclusively onto the regions of the object assigned to the corresponding component beams. This has the advantage that, in particular, object processing with a plurality of individual relatively small processing structures such as, for example, laser-bored holes can be carried out much more quickly by comparison with conventional laser processing methods. This is because the time for the processing of a subregion may be used by the deflecting unit not participating in this processing to the effect that this nonparticipating deflecting unit is precisely preadjusted such that the processing of a further structure is not delayed by travel paths and travel times of the previously nonparticipating deflecting unit.

The method for processing objects by use of laser beams may be used to bore at least one hole into the object to be processed. Given precise knowledge of the beam power of the primary laser beam, it is thereby possible to bore not only through holes, but also so-called blind holes with a high level of accuracy.

Use may be made as optical element of a disk silvered in subregions which is set rotating by a drive. The use of such a disk is advantageous, in particular, when precisely two component beams are used for the object processing. In this case, the disk preferably has a plane surface within which individual subregions produce precisely one reflected component beam, for example by vapor deposition of a thin silvered layer. Such a disk can preferably be arranged at an angle of 45° to the primary laser beam such that the beam path of the reflected further laser beam runs between the disk silvered in subregions and the further deflecting unit in a fashion exactly perpendicular to the beam path of the primary laser beam.

It may be pointed out at this juncture that it is also possible to use for the purpose of producing a plurality of component beams a plurality of silvered disks that are arranged one behind another in the beam path of the primary laser beam. It is thereby possible, given an appropriate synchronization of the rotations of the individual disks, to produce a plurality of spatially separate component beams with an approximately identical beam intensity in a simple way.

Rotation of the silvered disk may be synchronized with the repetition frequency of a pulsed laser. This has the advantage that no undesired reflections occur, particularly at the transitions between silvered and transparent regions of the silvered disk, and the nonuniform beam splitting is prevented at the same time. A nonuniform beam splitting would arise, for example, when the primary laser beam strikes precisely the transition between silvered and transparent regions.

### BRIEF DESCRIPTION OF THE DRAWINGS

Further advantages and features of the present invention emerge from the following exemplary description of a currently preferred embodiment.

In the drawings:

FIG. 1 shows a laser processing apparatus in accordance with an exemplary embodiment of the invention, and

FIG. 2 shows a plan view of the partially silvered disk illustrated in FIG. 1.

### DETAILED DESCRIPTION OF THE PREFERRED EMBODIMENTS

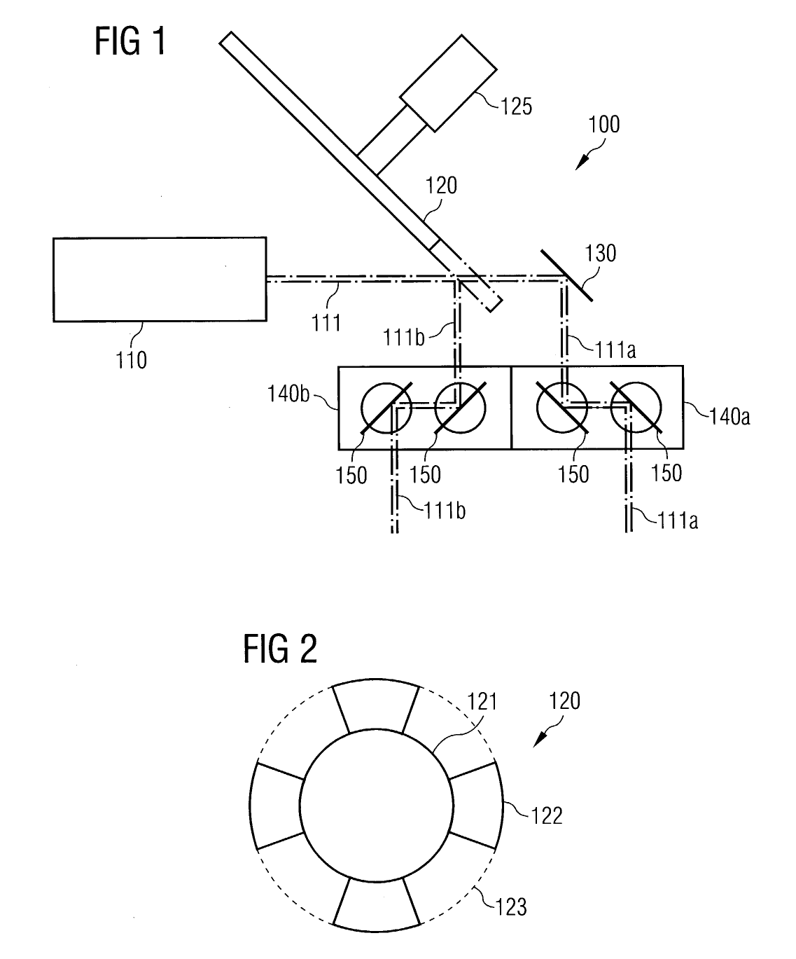

As may be seen from FIG. 1, a laser processing apparatus 100 has a laser 110 that produces a primary laser beam 111. This laser beam 111 strikes a chopper element 120 that is a disk silvered in subregions in accordance with the exemplary embodiment of the invention described here. The chopper element 120 is rotated about the rotation axis 126 by means of a servo drive 125.

Instead of the servo drive 125, it is also possible, of course, to use another type of motor or a drive mechanism by means of which the chopper element 120 can be rotated. Also conceivable is a periodic linear displacement of a reflector in the case of which, for example, a reflector is moved to and fro between two positions, the reflector being located in one position in the beam path, and in the other position outside the beam path of the primary laser beam 111.

The chopper element 120 is illustrated in a plan view in FIG. 2. The chopper element 120 has a basic element 121, silvered surfaces 122 and transparent surfaces 123. The silvered surfaces 122 and the transparent surfaces 123 are arranged alternately on the outer edge of the basic element 121. As may be seen from FIG. 2, the chopper element 120 in accordance with the exemplary embodiment illustrated here has four silvered surfaces 122 and four transparent surfaces 123. It may be pointed out that, of course, it is also possible to use chopper elements that have more or alternatively fewer silvered surfaces. It may be mentioned, furthermore, that it is also possible to fit only the silvered surfaces 122 on the basic element 121 such that, depending on the angular position of the chopper element 120, the primary laser beam 110 either strikes a silvered surface 122, or passes through between two neighboring silvered surfaces 122 without striking the chopper element 120.

As may be seen from FIG. 1, in accordance with the illustrated preferred exemplary embodiment of the invention the chopper element 120 is arranged at an angle of 45° relative to the primary laser beam 111. In accordance with the exemplary embodiment of the invention illustrated here, the rotation of the chopper element 120 leads to the fact that the primary laser beam 111 is either reflected at a silvered surface 122 or transmitted through a transparent surface 122, depending on the angular position of the chopper element 120. In this way, two component beams, a first transmitted laser beam 111a and a second reflected laser beam 111b, are produced sequentially. Because of the 45° angular position of the chopper element 120, the beam path of the second laser beam 111b runs initially perpendicular to the beam path of the primary laser beam 111. In accordance with the exemplary embodiment of the invention illustrated here, in order to produce two parallel laser beams 111a and 111b a reflector 130 is used that is likewise inclined by 45° to the primary laser beam 111 and thus leads to a beam deflection of the first laser beam 111a by 90° just like the silvered surfaces 122.

The first laser beam 111a strikes a first deflecting unit 140 by means of which the first laser beam 111a can be deflected in such a way that the laser beam 111a strikes the object to be processed (not illustrated) and processes it appropriately. The first deflecting unit 140 has two mirrors 150, the two mirrors 150 being mounted in each case rotatably about an axis, the axes of the two mirrors 150 being perpendicular to one another. The first laser beam 111a can thereby be deflected in such a way that it is possible to strike a two-dimensional region that is substantially perpendicular to the first laser beam 111a striking the object. The second laser beam 111b is deflected correspondingly by means of a second deflecting unit 140b that likewise has two mirrors 150 mounted rotatably perpendicular to one another. In this way, the second laser beam 111b can be used to process in a similar way a second subregion of the object (not illustrated).

It may be pointed out that the chopper element 120 splits the primary laser beam 111 into two laser beams 111a and 111b that alternately strike the object to be processed. This temporal beam splitting is advantageous particularly in the case of the processing of objects that are to be processed in a plurality of individual subregions separated from one another. It is possible in this way, for example, to use the first laser beam 111a to preset the second deflecting unit 140b during the processing of the object in such a way that the second laser beam 111b subsequently striking the object strikes the desired subregion of the object with high accuracy without delay because of an adjustment of the two mirrors 150 of the second deflecting unit 140b.

In summary, an embodiment of the invention creates a method and an apparatus for processing objects by means of laser beams. In this case, a primary laser beam 111 emitted by a laser 100 is split alternately by a so-called chopper element 120 into at least two component beams 111a and 111b with spatially different beam paths. Each of the two component beams 111a and 111b is guided onto the object to be processed in each case by means of a deflecting unit 140a and 140b, respectively. The temporal beam splitting of the primary laser beam 111 into the at least two component beams 111a and 111b has the advantage that in each case the deflecting unit 140b or 140a that is not being used at a specific instant, since precisely the other laser beam 111a or 111b is being used for processing the object, can be brought into a new predetermined position such that the subsequent laser beam 111a or 111b led via this deflecting unit strikes the desired subregion of the object without a time delay.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method for processing objects by use of laser beams, comprising:
directing a primary laser beam, produced by a laser, onto an optical element having at least one sequence of subregions, wherein the sequence includes a substantially transmitting first subregion and at least one substantially reflecting further subregion; and
varying the spatial position of the optical element in such a way that the primary laser beam is directed alternately onto the first subregion and onto the further at least one subregion, and thereby alternately producing a transmitting first laser beam and at least one further reflected laser beam, wherein the first laser beam is led to a first deflecting unit by which the first laser beam is guided onto an object to be processed in such a way that the object is processed in a first region, and wherein the further at least one laser beam is led to at least one further deflecting unit by which the further at least one laser beam is guided onto the object in such a way that the object is processed in at least one further region.

2. The method as claimed in claim 1, wherein an optical element is used that is designed in such a way that in its case, the further subregions are each plane reflecting surfaces which are arranged at an angle to one another.

3. The method as claimed in claim 1, wherein, during the processing of the first region by use of the first laser beam, the further deflecting unit is adjusted in such a way that the subsequently produced further laser beam is directed exclusively onto the further region of the object.

4. The method as claimed in claim 1, wherein during the processing of the further region by use of the further laser beam, at least one of
the first deflecting unit is adjusted in such a way that the subsequently produced first laser beam is directed exclusively onto the first region of the object, and
another further deflecting unit is adjusted in such a way that the subsequently produced other further laser beam is directed exclusively onto the other further region of the object.

5. The method as claimed in claim 1, wherein a hole is bored into the object to be processed by use of the first laser beam and the further laser beam.

6. The method as claimed in claim 1, wherein use is made as an optical element of a disk which is silvered in subregions and which is set rotating by use of a drive.

7. The method as claimed in claim 1, wherein a pulsed laser is used as laser.

8. The method as claimed in claim 7, wherein the rotation of the disk silvered in subregions is synchronized with the repetition frequency of the pulsed laser.

9. The method as claimed in claim 1, wherein the first laser beam is led to the first deflecting unit via an additional reflector.

10. An apparatus for processing objects using laser beams, comprising:
a laser, adapted to produce a primary laser beam;
an optical element, onto which the primary laser beam is directed, including at least one sequence of subregions, the sequence including a substantially transmitting first subregion and at least one substantially reflecting further subregion;
a drive, coupled to the optical element and adapted to vary the spatial position of the optical element in such a way that the primary laser beam alternately strikes the first subregion and the further at least one sub-region, to thereby alternately produce a transmitted first laser beam and at least one reflected further laser beam;
a first deflecting unit, adapted to direct the first laser beam onto a first region of an object to be processed; and
at least one further deflecting unit, adapted to direct at least one of the laser beam and the further laser beam onto a further region of the object to be processed.

11. The apparatus as claimed in claim 10, comprising:
a control unit, adapted to control the drive and the deflecting units in a synchronous fashion in such a way that,
during the processing of the first region by use of the first laser beam, the further deflecting unit is adapted to be adjusted in such a way that the subsequently produced further laser beam is guided exclusively onto the further region of the object, and
during processing of the further region by use of the further laser beam, at least one of
the first deflecting unit is adapted to be adjusted in such a way that the subsequently produced first laser beam is guided exclusively onto the first region of the object and
the other further deflecting unit is adapted to be adjusted in such a way that the subsequently produced other further laser beam is guided exclusively onto the other further region of the object.

12. The apparatus as claimed in claim 10, wherein the optical element is a disk silvered in subregions.

13. The apparatus as claimed in claim 10, further comprising:
a reflector, arranged in the beam path of the first laser beam between the optical element and the first deflecting unit.

14. The apparatus as claimed in claim 10, wherein the laser is a pulsed laser.

15. The apparatus as claimed in claim 10, wherein the at least one further subregion include at least two further subregions that are a plane reflecting surfaces that are arranged at an angle to one another.

16. The apparatus as claimed in claim 10, wherein the optical element includes at least two sequences with precisely one further subregion in each case, and precisely one further deflecting unit is provided, by which the precisely one further laser beam is adapted to be guided onto the precisely one further region of the object to be processed.

17. The method as claimed in claim 2, wherein, during the processing of the first region by use of the first laser beam, the further deflecting unit is adjusted in such a way that the subsequently produced further laser beam is directed exclusively onto the further region of the object.

18. The method as claimed in claim 2, wherein during the processing of the further region by use of the further laser beam, at least one of
the first deflecting unit is adjusted in such a way that the subsequently produced first laser beam is directed exclusively onto the first region of the object, and
another further deflecting unit is adjusted in such a way that the subsequently produced other further laser beam is directed exclusively onto the other further region of the object.

19. The method as claimed in claim 2, wherein a hole is bored into the object to be processed by use of the first laser beam and the further laser beam.

20. The method as claimed in claim 2, wherein use is made as an optical element of a disk which is silvered in subregions and which is set rotating by use of a drive.

21. The method as claimed in claim 2, wherein a pulsed laser is used as laser.

22. The method as claimed in claim 21, wherein the rotation of the disk silvered in subregions is synchronized with the repetition frequency of the pulsed laser.

23. The apparatus as claimed in claim 11, wherein the optical element is a disk silvered in subregions.

24. The apparatus as claimed in claim 11, further comprising:
a reflector, arranged in the beam path of the first laser beam between the optical element and the first deflecting unit.

25. The apparatus as claimed in claim 11, wherein the laser is a pulsed laser.

26. The apparatus as claimed in claim 11, wherein the at least one further subregion include at least two further subregions that are a plane reflecting surfaces that are arranged at an angle to one another.

27. The apparatus as claimed in claim 11, wherein the optical element includes at least two sequences with precisely one further subregion in each case, and precisely one further deflecting unit is provided, by which the precisely one further laser beam is adapted to be guided onto the precisely one further region of the object to be processed.

28. An apparatus for processing objects using laser beams, comprising:
laser means for producing a primary laser beam;
an optical element, onto which the primary laser beam is directed, including at least one sequence of subregions, the sequence including a substantially transmitting first subregion and at least one substantially reflecting further subregion;
drive means, coupled to the optical element, for varying the spatial position of the optical element in such a way that the primary laser beam alternately strikes the first subregion and the further at least one sub-region, to thereby alternately produce a transmitted first laser beam and at least one reflected further laser beam;
first deflecting means for directing the first laser beam onto a first region of an object to be processed; and
at least one further deflecting means for directing at least one of the laser beam and the further laser beam onto a further region of the object to be processed.

29. The apparatus as claimed in claim 28, comprising:
control means for controlling the drive and the deflecting means in a synchronous fashion in such a way that,
during the processing of the first region by use of the first laser beam, the further deflecting means is adapted to be adjusted in such a way that the subsequently produced further laser beam is guided exclusively onto the further region of the object, and
during processing of the further region by use of the further laser beam, at least one of
the first deflecting means is adapted to be adjusted in such a way that the subsequently produced first laser beam is guided exclusively onto the first region of the object and
the other further deflecting means is adapted to be adjusted in such a way that the subsequently produced other further laser beam is guided exclusively onto the other further region of the object.
